(19) United States
(12) Patent Application Publication
(10) Pub. No.: US 2019/0191678 A1
(43) Pub. Date: Jun. 27, 2019

(54) SYSTEM AND METHOD FOR BREEDING FLIES, PARTICULARLY BLACK SOLDIER FLIES (BSFs)

(71) Applicant: Faisal Emad Alrayya, Jeddah (SA)

(72) Inventor: Faisal Emad Alrayya, Jeddah (SA)

(21) Appl. No.: 16/292,392

(22) Filed: Mar. 5, 2019

(51) Int. Cl. A01K 67/033; A23K 10/20; A23K 50/90

(52) U.S. Cl. CPC A01K 67/033; A01K 2227/706; A23K 50/90; A23K 10/20

(57) ABSTRACT

Disclosed is a system and method for breeding flies, specifically Black Soldier Flies (BSFs) that can be used for bioconversion of organic waste into value added products such as fertilizers and animal feed. Particularly, the proposed system and method facilitate in breeding Black Soldier Flies (BSFs) and harvesting their larvae in a large scale.

### TECHNICAL FIELD

The present invention relates generally to an industrial method for breeding flies, more particularly to a system and method for breeding flies, specifically Black Soldier Flies (BSFs) that can be used for bioconversion of organic waste into value added products such as fertilizers and animal feed.

### BACKGROUND

Large amounts of organic waste are generated annually from agricultural plantations, animal farms, mills, food processing plants and industrial plants. The amount of organic waste generated has been increasing each year as the agro-food industries expand. These generated organic waste are a major cause of environmental pollution and are of less value to the human and animals.

Consequently, the safe disposal and proper utilization of these wastes has been a major concern in recent years. Different methods are available for safe disposal of these organic waste and for conversion of these waste into animal feed and fertilizers. The most acceptable methods for bioconversion of the organic waste into value added products often utilize microorganisms or insects such as different species of larvae of earthworms, house flies, or black soldier flies (BSFs) to efficiently treat the organic waste and produce high quality fertilizers and animal feed.

As the demand for such insects, particularly BSF larvae is increasing day by day, it is essential to mass produce or breed these insects/flies at an industrial level to meet ever increasing demand for such insects.

There are a number of characteristics of BSFs that have been considered advantageous in bioconversion processes due to which demand for BSFs is increasing. Particularly, BSFs larvae and pre-pupae are high in protein and fatty acid content and exhibit some natural behaviors that is very helpful and yield better results when these BSFs are utilized in the bioconversion process. In an experiment conducted, where 5000 BSFLs were placed into a container with 10 kg of organic wastes. The BSFLs managed to decompose more than 80% of the organic waste within a period of 5 days, which resulted in a reduced volume of the organic waste by 42% and weight by 70%. In comparison to the efficiency of house fly larvae and earthworm larvae widely used in the conventional treatment methods, the BSFLs are found to be more efficient in achieving the desired high value products such as fertilizers and animal feed. In contrast to the earthworm larvae and house fly larvae, the BSFLs spend much longer time decomposing the organic waste. Thus, culturing or reproducing these BSF larvae has become a necessity.

The BSF goes through full metamorphosis during its lifespan. The lifespan can be divided into egg, larval, pupae and adult life cycle stages. The larvae will hatch from the egg in about 4 days, and then the larvae will take about 2 weeks before they are ready to pupate (become adults). When they are ready to pupate they will try to find a dry sheltered area to bury themselves in before taking about 2 weeks to emerge as adult BSFs. Adult BSFs live for 5-8 days, this is all assuming that perfect conditions are present for breeding. The length of a Black Soldier Flies (BSF) life before it becomes an adult depends on several factors including weather conditions and how much food is available. BSFs in their pre-pupae larval stage are super-efficient in converting organic waste to nutrients (source: BSF Farming: blacksoldierflyfarming.com). In an appropriate weather conditions, the lifespan of BFSs are estimated to be approx. 37-41 days, thereby making them one of very suitable resource for bioconversion process.

A number of prior art discloses various methods and systems for production or breeding of BSFs. For example, U.S. Patent Application number 20110174222 describes about a specific breeding farm comprising a breeding net, induction medium, and a breeding induction apparatus having a plurality of breeding grooves, all of that facilitates breeding of Black Soldier Flies (BSF).

The U.S. Pat. No. 8,733,284 describes about an apparatus and method for breeding flying insects, particularly black soldier flies. The apparatus includes a cage and a covered bin. The bin contains a substrate (such as a high permeability/low water retention substrate such as perlite or vermiculite) where insect pupae develop into flies and then go to the cage for mating. The cage and the bin are provided with suitable water supply, air supply, and lighting means to facilitate the breeding process.

The Chinese Patent 103598148 describes a black soldier fly cultivation system and method. The system includes a pupation device and an adult black soldier fly cultivation chamber. The pupation device is used for incubating black soldier fly prepupa into black soldier fly pupa, and pupation media with water is disposed inside the pupation device to facilitate this incubation. The adult black soldier fly cultivation chamber is used for cultivating the black soldier fly pupa into adult black soldier fly.

The U.S. patent Ser. No. 10/159,229 describes apparatus and method for culturing Dipteran insects, particularly BSF. The apparatus in particular include a mating chamber to hold adult insects, and adaptations for various inputs such as: empty egg laying substrate, prepupae, bedding, carbohydrate, and various outputs such as egg laying substrate containing eggs, exuvia and bedding, and/or dead adults. The apparatus may also include adaptations to optimize the use of space including the use of controlled environmental conditions within a confined space.

Many other inventors also envisioned several methods and systems for mass-rearing of black soldier fly larvae (BSFL) or flies of like nature, some of those systems and methods are described in the US20180360008, U.S. Pat. No. 8,322,302, KR100654253, U.S. Pat. No. 9,462,795, KR101044636, and U.S. Pat. No. 5,351,643.

Despite various improvements and proposals, there exists a need for an improved and more reliable solution, particularly a system and method for massive culturing or breeding of the Black Soldier Flies (BSFs).

### SUMMARY

The present invention aims to provide a system and method for massive breeding of flies, particularly Black Soldier Flies (BSFs) and their larvae in an industrial scale.

Another object of the invention is to provide an improved method and system in which sanitary conditions can be maintained at all stages of the process for breeding of flies (particularly BSFs).

Another objective of the present invention is to provide a system and method for breeding Black Soldier Flies (BSFs) and harvesting their larvae for treatment or bioconversion of organic waste into value added products such as fertilizers and animal feed.

It is an objective of the present invention to provide a system and method for breeding and harvesting BSFs and their larvae in a large scale in a reduced amount of time with lesser human efforts.

Embodiments of the present invention discloses a system for breeding flies, particularly BSFs. The system includes a treatment chamber having an integral pathway leading to a first circumferential opening associated therewith, wherein the treatment chamber is configured to receive pre-pupae larvae, and hold feed material for the pre-pupae larvae to feed on, and undergo pupation; a dark chamber in communication with the treatment chamber, the dark chamber comprising a second circumferential opening, and a third circumferential opening, the third circumferential opening is selectively openable and closable by a gate controllable an actuator, wherein the dark chamber is configured to receive pupated larvae that crawl over the pathway via the first circumferential opening, and the second circumferential opening to reach therein, and wherein the dark chamber is further configured to permit the pupated larvae to transition into adult flies; a fly chamber in communication with the dark chamber, the fly chamber is configured to permit the adult flies to mate and oviposit eggs; an oviposition arrangement comprising of at least one an attractant holder configured onto a hatching chamber, and a plurality of bio-balls for receiving the eggs deposited therein by the adult flies, wherein the bio-balls are configured over a framework operatively rotatable to move the bio-balls from the fly chamber to the hatching chamber; wherein the hatching chamber is configured to facilitate hatching of the eggs contained inside the bio-balls to produce early stage larvae and collection of the early stage larvae that can then be harvested after a specific time and be used for decomposition of organic waste; and a central shaft with a rotating member mounted therein configured in a way to engage with the treatment chamber and the dark chamber, the central shaft when rotated facilitate internal cleaning of the treatment chamber and the dark chamber.

According to the embodiments, the treatment chamber is made semi-circular, hollow inside, and open at a first end, and a second end, and the dark chamber is made semi-circular, hollow inside, and open at a third end, and a fourth end. However, the dark ring is made smaller in diameter.

According to the embodiments, the fly chamber is made cylindrical and comprises at least one of: a light source positioned at a suitable location over the fly chamber and selectively activated to act on the adult flies to urge the adult flies to leave the dark chamber and spend rest of their lifespan in the fly chamber for matting; a vibration source positioned at a suitable location over the fly chamber and selectively activated to act on the adult flies to urge the adult flies to participate in matting for higher yield of the eggs; and a plant like motivation element positioned at a suitable location inside the fly chamber to urge the adult flies to sit thereon and mate. The fly chamber is made cylindrical in shape and made of wire mesh material to prevent the flies from escaping away. The fly chamber is further lined with a transparent screen from outside.

According to the embodiments, each of the treatment chamber, the dark ring, the fly chamber, the hatching chamber are supplied with at least one of: air circulation provision, water circulation provision, and feed material supplying provision in order to maintain suitable atmospheric conditions therein to facilitate breeding and harvesting of larvae.

According to the embodiments, the hatching chamber is half-moon shaped. The hatching chamber is further provided with a brush configured thereon to remove the flies present over the bio-balls when the bio-balls are rotated from the fly chamber to the hatching chamber.

These and other objects of the present invention will become readily apparent upon further review of the following specification and drawings.

### BRIEF DESCRIPTION OF DRAWINGS

The foregoing summary, as well as the following detailed description of preferred embodiments, is better understood when read in conjunction with the appended drawings. There is shown in the drawings example embodiments, however, the application is not limited to the specific system and method disclosed in the drawings.

FIG. 1 shows a system for breeding flies, according to an embodiment of the present invention.

FIG. 2 shows a top view of the system shown in FIG. 1.

FIG. 3 shows a wired diagram of the system shown in FIG. 1.

FIG. 4 shows a sectional view of the system shown in FIG. 1 and FIG. 3 along A-A′, according to an embodiment of the present invention.

FIG. 5 shows a treatment chamber, according to an embodiment of the present invention.

FIG. 6 shows a dark chamber, according to an embodiment of the present invention.

FIG. 7 shows a fly chamber, according to an embodiment of the present invention.

FIG. 8 shows a hatching chamber with an oviposition arrangement configured therein, according to an embodiment of the present invention.

FIG. 9 shows a central shaft with a member configured in a way to engage with the treatment chamber and the dark chamber, according to an embodiment of the present invention.

FIG. 10 shows a gate controllable by an actuator for opening and closing of third circumferential opening present in the dark chamber in order to permit movement of pupated larvae from the treatment tower to the dark tower, according to an embodiment of the present invention.

FIG. 11 shows bio balls configured over a framework operatively rotatable to move the bio-balls from the fly chamber to the hatching chamber, according to an embodiment of the present invention.

FIG. 12 shows a wired diagram of an alternative embodiment of the system shown in FIG. 3.

FIG. 13 shows an alternative hatching chamber, according to another embodiment.

### DETAILED DESCRIPTION

Various embodiments will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although any methods, and systems similar or equivalent to those described herein can be used in the practice or testing of embodiments, the preferred methods, and systems are now described. The disclosed embodiments are merely exemplary.

References to “one embodiment”, “an embodiment”, “another embodiment”, “an example”, “another example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment. Unless stated otherwise, terms such as “first”, “second”, “third”, “fourth”, are used to arbitrarily distinguish between the elements such terms describe. Thus, these terms are not necessarily intended to indicate temporal or other prioritization of such elements.

Described herein are systems and methods for breeding insects/flies particularly Black Soldier Flies (BSFs) that can be used for bioconversion of organic waste into value added products such as fertilizers and animal feed. The system and method disclosed herein allow for the breeding and harvesting of the BSF, also known as Hermetia illucens, and their larvae. However, the disclosed system and method are not limited to this insect species and, according to some embodiments, may be applied to other insect species as well.

The described system and method facilitates the full lifecycle of the BSFs and their larvae, and attempts to automate the lifecycle on a commercial scale. The system disclosed herein for breeding is capable of being scaled up for larger production of the BSFs.

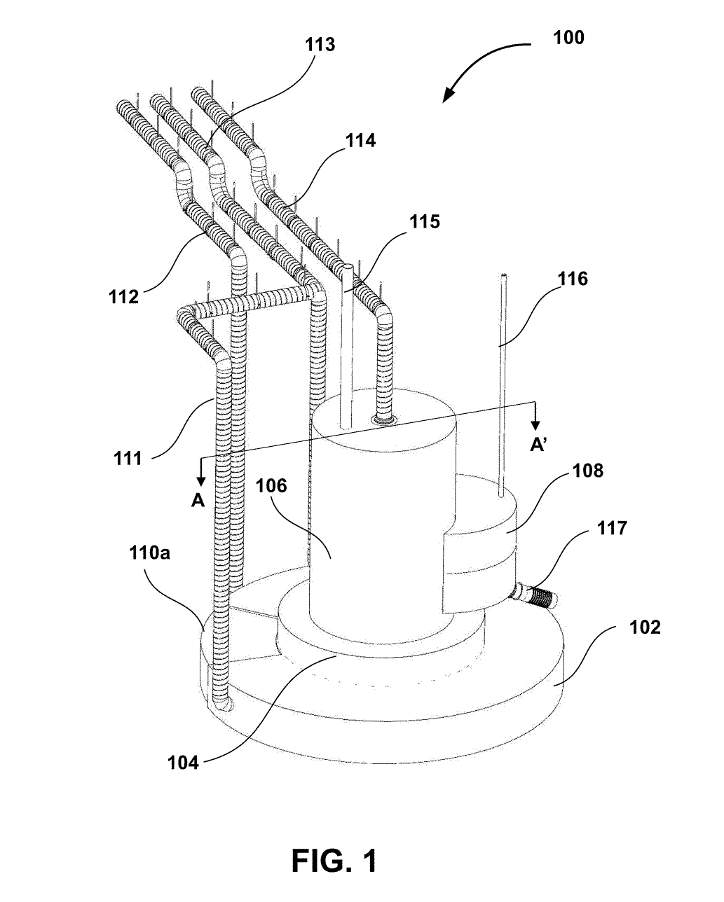

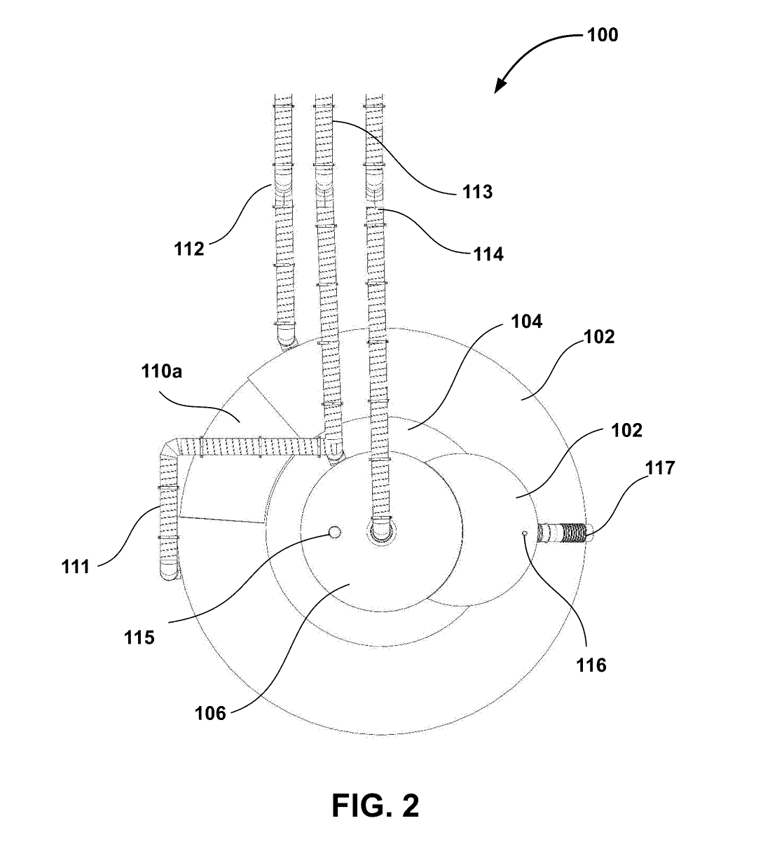

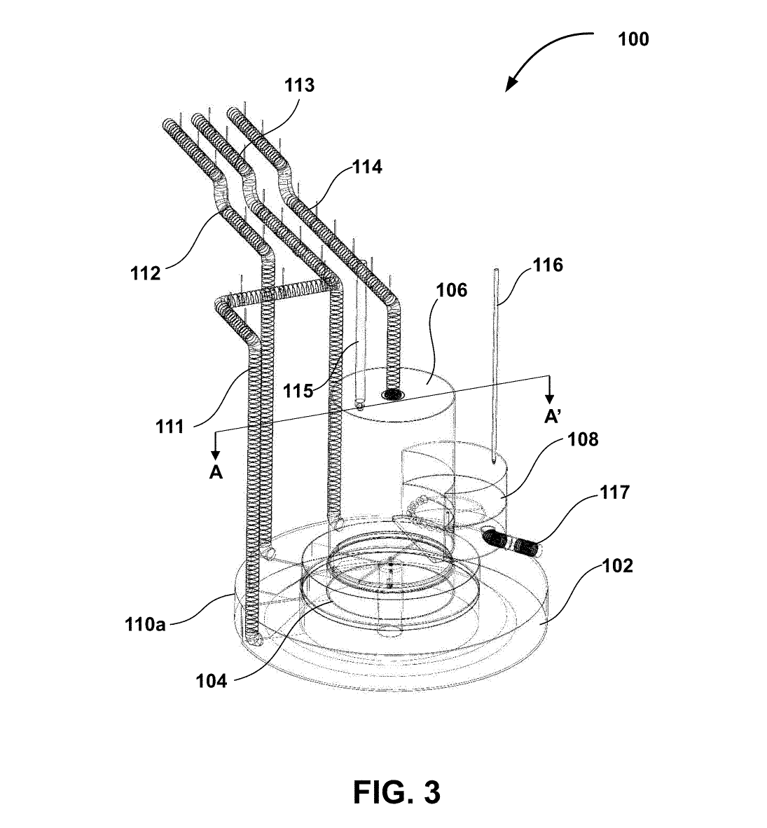

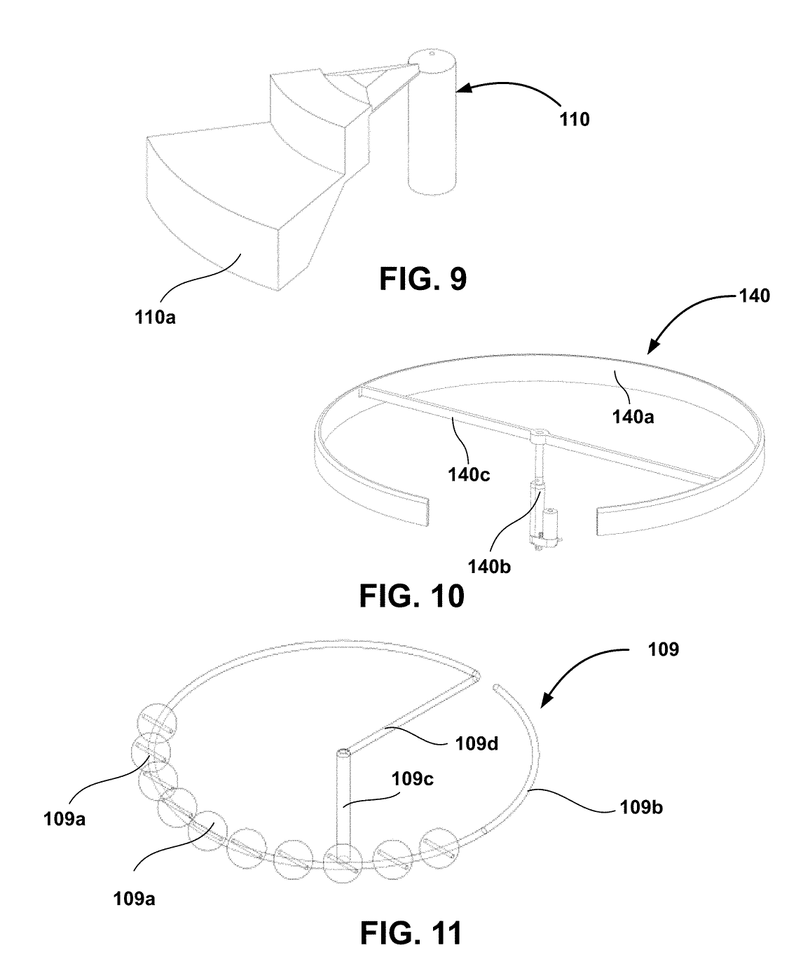

The various features and embodiments of the present system and method for breeding flies, particularly the BSFs are better explained in conjunction with FIGS. 1-11.

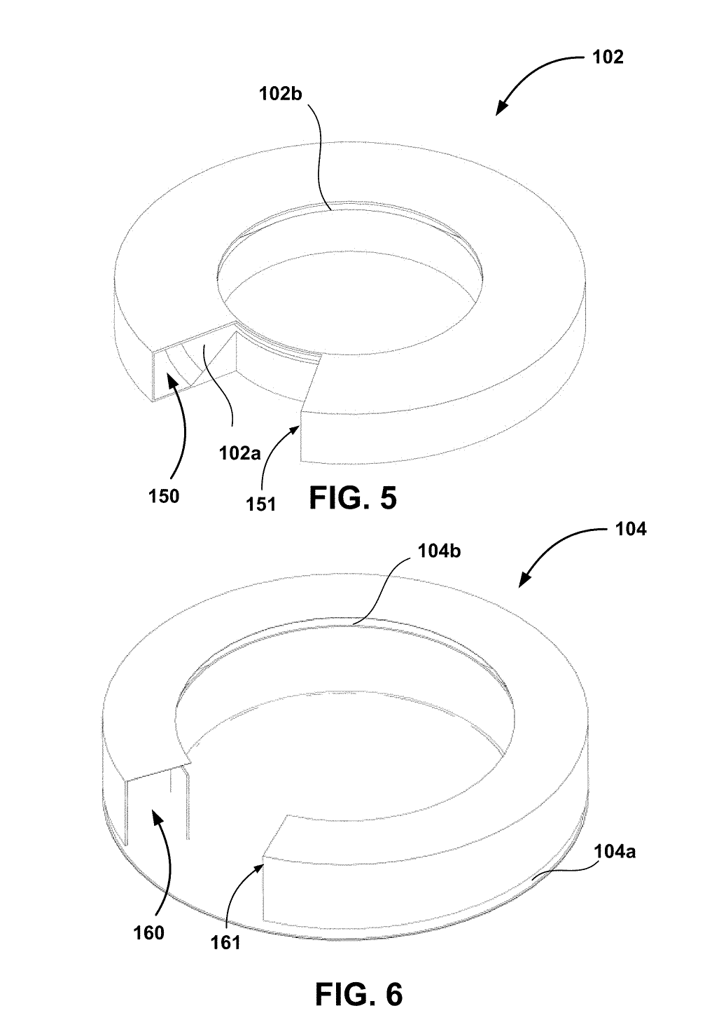

Referring to accompanying figures, particularly the FIGS. 1-4, the system 100 primarily includes a treatment chamber 102, a dark chamber 104, a fly chamber 106, and a hatching chamber 108 all operationally interconnected to each other to facilitate breeding of BSFs. The treatment chamber 102 as best shown in the FIG. 5, is preferably made circular or semi-circular in shape. The treatment chamber 102 may be made of any suitable metallic or non-metallic material. The treatment chamber 102 is made hollow inside and include two open ends 150, and 151. The treatment chamber 102 further includes an integral pathway 102a that leads to a first circumferential opening 102b associated therewith. The pathway 102a is inclined at a predetermined angle. In an example, the predetermined angle is about 40 degrees. The circumferential opening 102b associated with the treatment chamber 102 is of a predetermined width. In an example, the predetermined width is about 1″.

The treatment chamber 102 is configured to receive pre-pupae larvae or early stage pre-pupae larvae, and receive feed material for the pre-pupae larvae to feed on, and undergo pupation. The feed material preferably include but not limited to organic food material such as rotten vegetables. The feed material and the early stage pre-pupae larvae may be disposed into the treatment chamber 102 using several known techniques, however in the light of present invention, the feed material and the early stage pre-pupae larvae may be disposed or deposited inside the treatment chamber 102 using pumping systems known in the art.

Further to facilitate proper weather conditions within the treatment chamber 102, the treatment chamber 102 may include provisions such as one or more passageways, for example passageways 111 to pump in/channel in air inside the treatment chamber 102, and passageways 112 to pump out/channel out air from the treatment chamber 102. This is necessary in order to maintain adequate level of moisture inside the treatment chamber 102 so that the early stage pre-pupae larvae can actively consume the feed material. The treatment chamber 102 further facilitate the pre-pupae larvae to develop into pupae in the provided suitable weather conditions therein. The passageways 111 and 112 may be provided with mesh filters to prevent escaping out of any pre-pupae larvae through those passageways.

The system 100 further includes the dark chamber 104 in communication with the treatment chamber 102. The dark chamber 104 as best shown in the FIG. 6, is preferably made circular or semi-circular in shape. The dark chamber 104 may be made of any suitable metallic or non-metallic material. The dark chamber 104 is made hollow inside and include two open ends 160, and 161. The dark chamber 104 includes a second circumferential opening 104a, and a third circumferential opening 104b associated therewith. The circumferential opening 104a associated with the dark chamber 104 is of a predetermined width, preferably equal (but not necessary) in width to the first circumferential opening 102b of the treatment tower 102. The width of the circumferential opening 104a is preferably made equivalent to the width of the circumferential opening 102b so that during the engagement of the treatment chamber 102 and the dark chamber 104, those two openings 102b, 104a can coincide with each other to form a common passageway leading to the dark chamber 104. The width of the circumferential opening 104a should be made suitable such that it forms a passageway and can allow pupated larvae from the treatment chamber 102 to crawl and get into the dark chamber 104. Specifically, allow the pupated larvae to crawl over the pathway 102a via the first circumferential opening 102b, and the second circumferential opening 104a to reach the dark chamber 104. It is the dark chamber 104 that permit the pupated larvae to transition into adult flies. The dark chamber 104 is configured to be very dark (without any light) and dry that helps in the transition of the pupated larvae to the adult flies. The third circumferential opening 104b of the dark chamber 104 is of a predetermined width. The width of the circumferential opening 104b may or may not be equivalent to the width of the circumferential opening 104a. In an example, the width of the circumferential opening 104b is 1.5″.

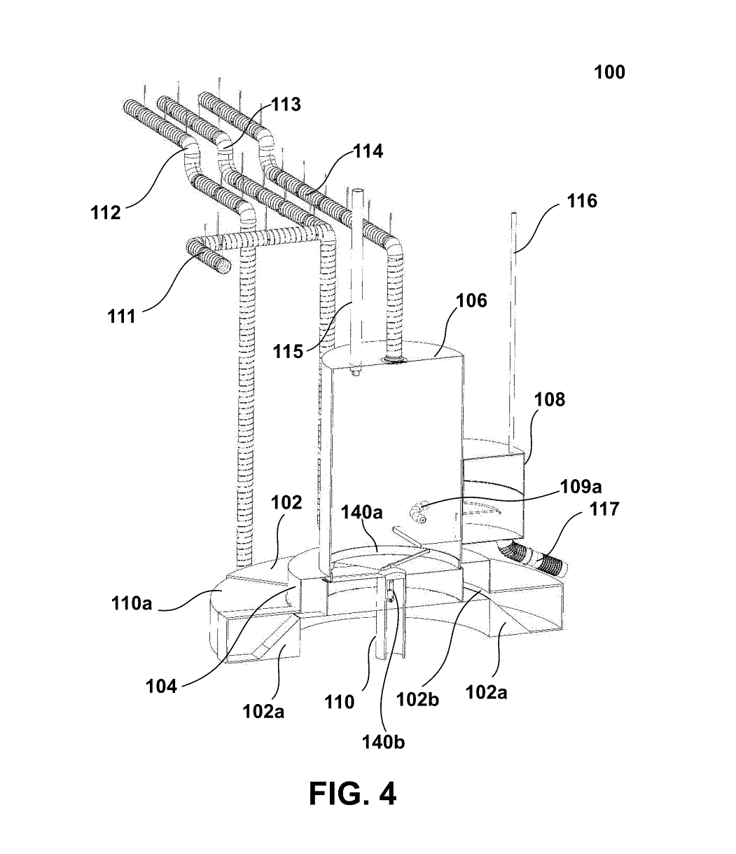

The circumferential opening 104b is selectively openable and closable by a gate 140 that's controllable by an actuator 140b. The actuator 140 may be an electrical actuator or a hydraulic actuator. As seen in the FIG. 10, the gate 140 includes a ring member 140a, semi-circular in shape complementing to the shape of the dark ring 104. The width of the ring member 140a is made equal to the width of the circumferential opening 104b. The actuator 140b is connected to the ring member 140a using a shaft 140c. According to the embodiment, and as best shown in the FIG. 4, the actuator 140b is housed or disposed within a central shaft 110. Further, the actuator 104b nay be provided with one or more bearings to ensure the gate 140, particularly the ring member 140a doesn't rotate when the central shaft 110 is made to rotate for rotating the rotating member 110a. The central shaft 110 is partially or fully opened on top to facilitate the operation of the actuator 140b disposed therein. The central shaft 110 includes a rotating member 110a mounted thereon and configured in a way to engage with the treatment chamber 102 and the dark chamber 104. The engagement of the rotating member 110a with the treatment chamber 102 and the dark chamber 104 completes the semi-circular shapes of the treatment chamber 102 and the dark chamber 104. The central shaft 110 when rotated using a motor (not shown) facilitate internal cleaning of the treatment chamber 102 and the dark chamber 104 to rinse of any remaining waste or died flies or larvae present therein. In operation, the two ends 150,151 of the treatment chamber 102, and the two ends 160, 161 of the dark chamber 104 are open and thus facilitate the solid rotating member 110 to rotate therein when the central shaft 110 is rotated.

Besides being dark and dry, the dark ring 104 according to the embodiments may also require an appropriate atmospheric condition inside for the pupated larvae to convert to the flies. The atmospheric condition inside the dark chamber 104 may be maintained using air supply provisions or one or more passageways, such as 113 for channeling in/pumping in air inside the dark chamber or pumping out/channeling out the air out of the dark chamber 104. The passageway 113 may be provided with mesh one or more filters to prevent escaping out of any adult flies through those passageway.

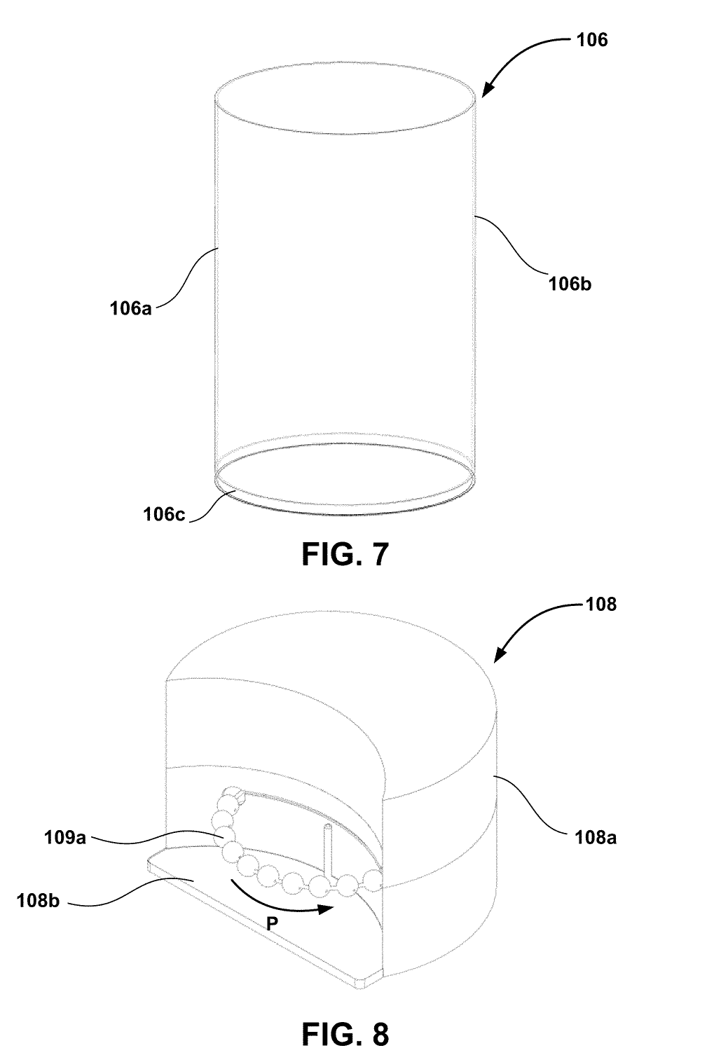

In operation, when the pupated larvae is converted to the adult flies, the actuator 140b may be activated to move the piston in upward direction and open the gate 110 (that's initially in closed state). This would allow the adult flies to fly out of the dark chamber 104 and be collected into a fly chamber 106 in communication with the dark chamber 104. According to the embodiment, an additional light source (not shown) may be positioned at a suitable location over the fly chamber 106 may be selectively activated to act on the adult flies and urge them to leave the dark chamber 104 and fly into the fly chamber 106 for mating and spending rest of their lifecycle. In an example, the light source is a LED light source. In another example, the light source may be halogen lamps readily available in the market. Although not explicitly shown, the light source may preferably be configured on top of the fly chamber 106. As shown in the FIG. 7, the fly chamber 104 may have an opening or spacing 106c at bottom to allow the flies from the dark chamber 104 to fly into the fly chamber 106. When in the fly chamber 106, the flies usually don't require any feed material and only water supply is sufficient to keep them lively. Thus, one or more passageways may be used, such as passageways 114, 115 for supplying water, and also for pumping in air and out of the fly chamber 106. The passageways 114 and 115 may be provided with one or more mesh filters to prevent escaping out of any flies through those passageways.

According to various embodiment, the fly chamber 106 may be provided with a vibration source (not shown) positioned at a suitable location over the fly chamber 106 that can be selectively activated to act on the adult flies to urge them to participate in matting for higher yield of the eggs. In an example, the vibration source is an audio speaker that can play music. Further, according to the embodiment, the fly chamber 106 may further be provided with a plant like motivation element (not shown) positioned at a suitable location inside the fly chamber 106 (on top preferably) to urge the flies to sit thereon and mate.

The fly chamber 106 is cylindrically shaped and made of wire mesh material 106a sufficient enough to prevent the flies from escaping away. Further, the fly chamber may 106 be lined with a transparent screen 106b from outside. The wire mesh material 106a may be metallic or non-metallic. The transparent screen 106b adapted for covering the wire mesh material 106a may be made of thin plastic material. The transparency in the screen 106b may assist in visualizing internal environment conditions within the fly chamber 106. Further, the transparent screen 106b may also help with letting the adequate sunlight enter inside the fly chamber 106, which is essential for this stage of their lifecycle. The fly chamber 106 is configured to permit the adult flies to mate and oviposit eggs utilizing an oviposition arrangement.

In the context of the present invention, the oviposition arrangement refers to arrangements or components that in general help the flies in laying eggs. According to the embodiment, the oviposition arrangement includes an attractant holder 108b configured onto a hatching chamber 108 or one that forms a part of the hatching chamber 108 as shown in the FIG. 8. The attractant holder 108b according to an embodiment holds attractant (not shown). The attractant may include but not limited to the feed material and antibiotics added therein to preserve the feed material for a longer duration. At this stage, the flies have strong senses, thus the flies are attracted towards this attractant for laying eggs (oviposition), and this also helps the flies to fly towards the region of attractant and sit and lay eggs on a plurality of bio-balls configured over top of the attractant holder 108b.

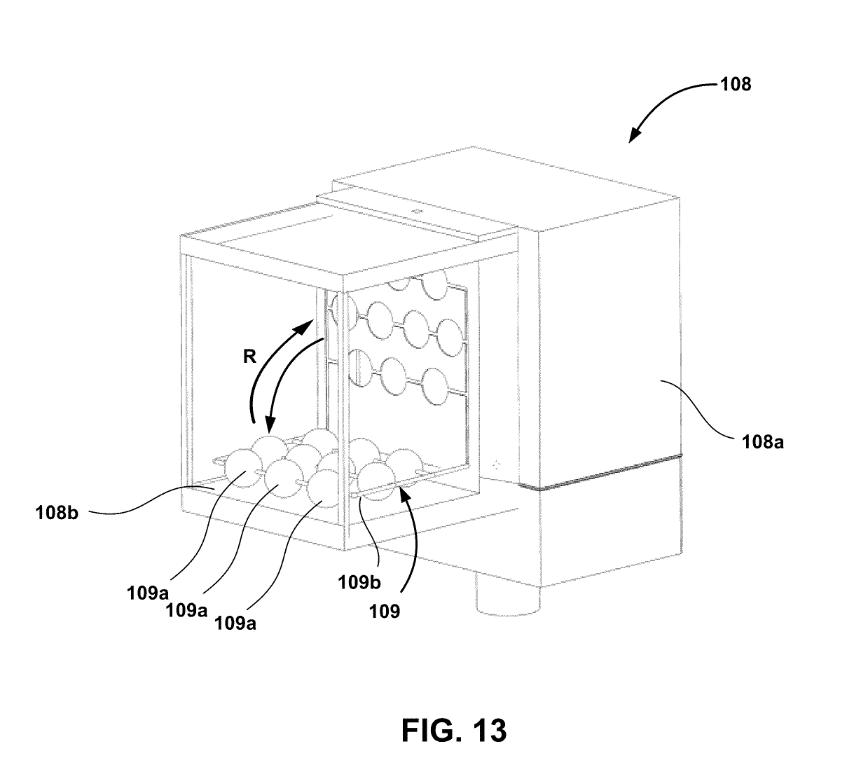

The bio-balls 109a forming a part of the oviposition arrangement, such as bio-balls 109a (as shown in the FIG. 11) is configured to receive the eggs deposited by the adult flies. The bio-balls 109a may be made of any suitable materials, such as but not limited to wood. The bio-balls 109a are configured in but not limited to circular shape with apertures distributed over their surface so that the adult flies can lay eggs into the balls 109a. The bio-balls 109a are preferably stuffed with sponge or like soft material for preventing any accidental damage to the eggs deposited therein. The balls 109a are mounted over a framework 109b. The framework 109b is coupled to an L-member 109c, 109d (configured as two pieces attached to each other or as a one piece item). The L-member 109c, 109d may be selectively operated to rotate the framework 109b, which in turns rotate the bio-balls 109a mounted thereon. In operation, once the bio-balls or balls 109a has eggs deposited therein, then the framework 109b may be rotated using a motor (not shown) preferably a step motor in 180 degrees to move the bio-balls 109a from the fly chamber 106 to the hatching chamber 108. This rotation is along horizontal plane along the direction P. The hatching chamber 108 is further provided with a brush (not shown) configured to remove the flies present over the bio-balls 109a when the bio-balls 109a are rotated from the fly chamber 106 to the hatching chamber 108. In the preferred embodiment, the L-member 109c, 109d and the framework 109b are configured to rotate the bio-balls 109a along a horizontal plane, it should be understood that the framework 109b may be suitably configured to rotate the bio-balls 109a along a vertical plane along the direction ‘R’ as shown in the FIG. 13.

The hatching chamber 108 is configured to facilitate hatching of the eggs contained inside the bio-balls 109a to produce early stage larvae, which may be collected and maintained in a hollow hatch box region 108a of the hatching chamber 108. Like other chambers, namely the treatment chamber 102, the dark chamber 104, the fly chamber 106, it is also essential to maintain appropriate atmospheric conditions suitable for the growth of the early stage larvae. According to the embodiments, the hatching chamber 108 may be supplied by a source of diet food that's required for the growth of the early stage larvae and may be provided with air circulation provisions (one or more passageway) such as passageway 116 to maintain the perfect atmospheric condition for the growth of the larvae. The passageway 116 may be provided with one or more mesh filter to prevent escaping out of any flies through the passageway. The larvae after hatching will feed in the hatch box region 108a for a couple of days or so until water start to spray on the surface of the box 108a. Once the larvae has spent desired number of days and evolved into appropriate larvae preferably 5 days old larvae (DoL), an ejector pump 117 at the bottom of the hatching chamber 108 may be opened to transfer the 5 DoL to a shaker/separator means where the 5 DoL will be separated and suitably stored for use in conversion of the organic waste to the value added product such as quality fertilizers or animal feed. According to the embodiment, the hatching chamber 108 is preferably half-moon shaped. According to some other embodiment, the hatching chamber 108 may be made square in shape. Although, the embodiment of present invention is describes harvesting of larvae out of the hatching chamber 108 when they are 5 days old, it should be understood that within the scope of the scope of present invention, even larvae of 3 days old, 4 days old, 6 days old, 7 days old etc. can also be harvested, it is just that, 5 days old larvae (SDoL) is capable of yielding better results to act on the organic waste to convert them into value added products.

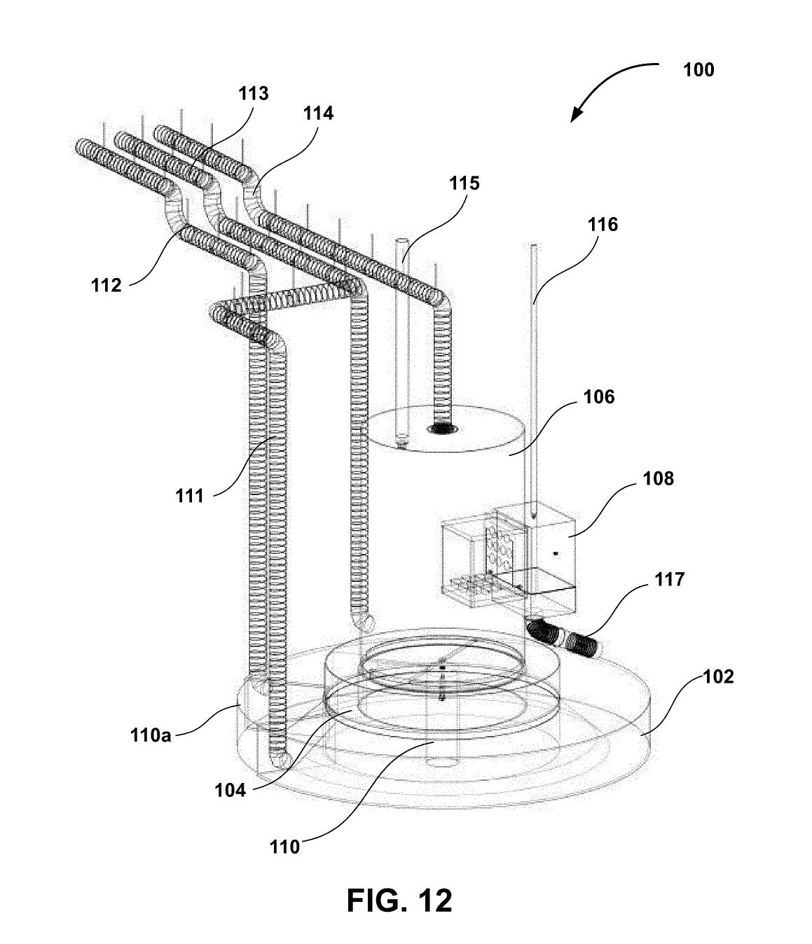

Referring to the FIGS. 12-13, an alternative embodiment of the system 100 is shown. Particularly, the elements/components shown in the FIG. 12 are identical to the components shown and described with respect to the FIGS. 1-11, except the hatching chamber 108. As described above with respect to the preferred embodiment in the FIGS. 1-11, the hatching chamber 108 includes a hollow hatch box region 108a (squared shaped), an attractant holder 108b, a plurality of bio-balls 109a, and a framework 109b. The bio-balls 109a are mounted on the framework 109b and are rotates (using a step motor) along the vertical plane to move the balls 109a containing eggs from the fly chamber 106 to the hatching chamber 108.

It should be noted that, the breeding system and associated components described above in the FIGS. 1-11 may be made using any suitable materials known in the art, and be made in different other suitable shapes and dimensions that may make the presented invention realization in real scenario.

The preceding description has been presented with reference to various embodiments. Persons skilled in the art and technology to which this application pertains will appreciate that alterations and changes in the described structures and methods of operation can be practiced without meaningfully departing from the principle, spirit and scope of the present invention.

What is claimed is:

1. A system (100) for breeding flies, comprising:
a treatment chamber (102) having an integral pathway (102a) leading to a first circumferential opening (102b) associated therewith, wherein the treatment chamber (102) is configured to receive pre-pupae larvae, and hold feed material for the pre-pupae larvae to feed on, and undergo pupation;
a dark chamber (104) in communication with the treatment chamber (102), the dark chamber (104) comprising a second circumferential opening (104a), and a third circumferential opening (104b), the third circumferential opening (104b) is selectively openable and closable by a gate (140) controllable an actuator (140b), wherein the dark chamber is configured to receive pupated larvae that crawl over the pathway (102a) via the first circumferential opening (102b), and the second circumferential opening (104a) to reach therein, and wherein the dark chamber (104) is further configured to permit the pupated larvae to transition into adult flies;
a fly chamber (106) in communication with the dark chamber (104), the fly chamber (104) is configured to permit the adult flies to mate and oviposit eggs;
an oviposition arrangement comprising of at least one an attractant holder (108b) configured onto a hatching chamber (108), and a plurality of bio-balls (109a) for receiving the eggs deposited therein by the adult flies, wherein the bio-balls (109a) are configured over a framework (109b) operatively rotatable to move the bio-balls (109a) from the fly chamber (106) to the hatching chamber (108);
wherein the hatching chamber (108) is configured to facilitate hatching of the eggs contained inside the bio-balls (109a) to produce early stage larvae and collection of the early stage larvae that can then be harvested after a specific time and be used for decomposition of organic waste; and
a central shaft (110) with a rotating member (110a) mounted therein configured in a way to engage with the treatment chamber (102) and the dark chamber (104), the central shaft (104) when rotated facilitate internal cleaning of the treatment chamber (102) and the dark chamber (104).

2. The system (100) of claim 1, wherein the treatment chamber (102) is semi-circular, hollow inside, and open at a first end (150), and a second end (151).

3. The system (100) of claim 1, wherein the dark chamber (104) is semi-circular, hollow inside, and open at a third end, and a fourth end.

4. The system (100) of claim 1, wherein the pathway (102a) is inclined at a predetermined angle.

5. The system (100) of claim 4, wherein the predetermined angle is about 40 degrees.

6. The system (100) of claim 1, wherein the flies comprises Black Soldier Flies (BSFs).

7. The system (100) of claim 1, wherein the fly chamber (106) further comprising at least one of:
a light source positioned at a suitable location over the fly chamber (106) and selectively activated to act on the adult flies to urge the adult flies to leave the dark chamber (104) and spend rest of their lifespan in the fly chamber (106) for matting;
a vibration source positioned at a suitable location over the fly chamber (106) and selectively activated to act on the adult flies to urge the adult flies to participate in matting for higher yield of the eggs; and
a plant like motivation element positioned at a suitable location inside the fly chamber (106) to urge the adult flies to sit thereon and mate.

8. The system (100) of claim 7, wherein the fly chamber (106) is cylindrically shaped and made of wire mesh material to prevent the flies from escaping away.

9. The system (100) of claim 8, wherein the fly chamber (106) is further lined with a transparent screen from outside.

10. The system (100) of claim 7, wherein the vibration source is an audio speaker.

11. The system (100) of claim 7, wherein the light source is a LED light source.

12. The system (100) of claim 1, wherein the attractant holder holds attractant comprising of the feed material and antibiotics added therein to preserve the feed material for a longer duration.

13. The system (100) of claim 12, wherein the attractant facilitates in oviposition.

14. The system (100) of claim 1, wherein the bio-balls (109a) are stuffed with soft material for preventing any accidental damage to the eggs deposited therein.

15. The system (100) of claim 1, wherein the actuator (140b) comprises at least one of: an electrical actuator and a hydraulic actuator.

16. The system (100) of claim 1, wherein the central shaft (110) is partially or fully opened on top and configured to hold and facilitate the actuator (140b).

17. The system (100) of claim 1, wherein the hatching chamber (108) is at least one half-moon shaped or square shaped.

18. The system (100) of claim 1, wherein each of the treatment chamber (102), the dark ring (104), the fly chamber (106), the hatching chamber (108) are supplied with at least one of: air circulation provision, water circulation provision, and feed material supplying provision in order to maintain suitable atmospheric conditions therein to facilitate breeding and harvesting of larvae.

19. The system (100) of claim 1, wherein the hatching chamber (108) is further provided with a brush configured thereon to remove the flies present over the bio-balls (109a) when the bio-balls (109a) are rotated from the fly chamber (106) to the hatching chamber (108).

20. The system (100) of claim 1, wherein the framework (109b) is operatively rotated to move the bio-balls (109a) from the fly chamber (106) to the hatching chamber (108) in at least one horizontal place or vertical plane.
